(19) United States
(12) Patent Application Publication
(10) Pub. No.: US 2022/0024950 A1
(43) Pub. Date: Jan. 27, 2022

(54) METHOD FOR PREPARING BORIC ACID ESTER BASED ON LITHIUM COMPOUND

(71) Applicant: SOOCHOW UNIVERSITY, Suzhou (CN)

(72) Inventors: Mingqiang XUE, Suzhou (CN); Xiaojuan XU, Suzhou (CN); Dandan YAN, Suzhou (CN)

(21) Appl. No.: 17/413,521

(22) PCT Filed: Mar. 7, 2019
(86) PCT No.: PCT/CN2019/077386

§ 371 Date: Jun. 11, 2021

(30) Foreign Application Priority Data
Dec. 12, 2018 (CN) 201811520091.6
Dec. 12, 2018 (CN) 201811521227.5
Dec. 18, 2018 (CN) 201811555577.3
Dec. 18, 2018 (CN) 201811555578.8

(51) Int. Cl. C07F 5/04; C07C 29/09; B01J 31/02

(52) U.S. Cl. CPC C07F 5/04; B01J 31/0252; C07C 29/095

(57) ABSTRACT

A method for preparing the borate ester using a lithium compound includes: under the inert gas, stirring and mixing carboxylic acid and borane, and a catalyst lithium compound is added, then the borate ester is obtained with hydroboration; wherein the hydroboration is at room temperature for 10 to 80 min. After the hydroboration and is stopped by contacting air, the solvent is removed under reduced pressure, to obtain the borate esters with different substituents. The lithium compounds are n-butyl lithium, lithium aniline, p-methyl lithium aniline, o-methyl lithium aniline, 2-methoxyaniline lithium, 4-methoxyaniline lithium, 2,6-dimethylaniline lithium, and 2,6-diisopropylaniline lithium. The lithium compounds disclosed in the present invention can catalyze the boron hydrogenation reaction of carboxylic acid and borane with high activity under room temperature conditions; the amount of lithium compound is 0.1-0.9% of the molar amount of carboxylic acid.

### TECHNICAL FIELD

The invention relates to the field of a commercial reagent lithium compound, and in particular, relates to a method for preparing the borate ester using the lithium compound.

### BACKGROUND ART

Organic boronic acid esters can be regarded as derivatives in which the hydrogen in orthoboric acid B(OH)₃ is replaced by organic groups, in addition to metaborate (ROBO)₃. Due to its stability and low toxicity, borate is widely used in various fields. It is a main raw material for the synthesis of boron-containing compounds. Borate compounds can be used not only as rust inhibitors, preservatives, polymer additives, anti-wear additives, automobile brake fluids, gasoline additives, flame retardants, but also as lubricant additives.

Existing borohydride methods have obvious shortcomings: LiAlH₄ and NaBH₄ systems have great safety risks, the SmI₂—H₂OEt₃N system requires a lot of excess reagents, and the Ru, Rh, Ir, Co transition metal complex systems require high temperature and high pressure. On one hand, it is difficult to synthesize the catalyst, and involves a high cost; on the other hand, the catalytic reaction requires a reaction temperature of 60° C. and a reaction time of 24 hours. The difference between the nucleophilic addition reaction activity of carboxylic acid and aldehyde and ketone: (1) the active hydrogen of carboxylic acid is easy to leave, so its two O's are actually equivalent, so from the perspective of steric structure, the steric hindrance of carboxyl group is more Large, and the existence of carboxyl hydrogen bonds makes the electron cloud density of the entire carbonyl group larger, and it is more difficult for nucleophiles to attack the active center; (2) Nucleophiles attack the carbonyl carbon first, which is related to the electron cloud density on the carbon. Aldehydes and ketones have a smaller electron cloud density than esters and amides, so they are highly active and will react preferentially. Those with large steric effects are not easy to react; (3) When forming a transition state, it depends on the leaving group, aldehyde The leaving groups of ketones are alkyl and hydrogen, both of which are not easy to leave. Therefore, aldehydes and ketones only undergo addition without elimination, which is different from carboxylic acids and their derivatives. On the one hand, the existing methods use catalysts that are difficult to synthesize and cost high; on the other hand, the catalytic reaction requires a reaction temperature of 60° C. and a reaction time of 24 hours.

### Technical Problems

This invention is in order to supply a method for preparing the borate ester based on lithium compound, that is, lithium compound is used as an efficient catalyst to catalyze the borohydride reaction of carboxylic acid and borane. The carboxylic acid is generally solid, and the carboxylic acid and pinacol. The reaction of borane is a heterogeneous reaction.

### Technical Solutions

In order to achieve the above purposes, the technical proposal adopted by the invention is: A method for preparing the borate ester based on lithium compound comprising the following steps: under the inert gas, stirring and mixing carboxylic acid and borane, and a catalyst lithium compound is added, then the borate ester is obtained with hydroboration; the lithium compound is n-butyllithium, anilinolithium, p-toluidine lithium, o-methylanilinide lithium, 2-methoxyanilinide lithium, 4-methoxy lithium anilinide, lithium 2,6,-dimethylanilinide, or lithium 2,6-diisopropylanilinide.

A method for preparing an alcohol compound based on lithium compound includes the following steps: under the inert gas, stirring and mixing carboxylic acid and borane, and a catalyst lithium compound is added, then the borate ester is obtained with hydroboration; after hydroboration adding silica gel and methanol, the alcohol compound is obtained after hydrolysis reaction.

Application of lithium compound in catalyzing hydroboration of carboxylic acid and borane; the lithium compound is n-butyllithium, anilinolithium, p-toluidine lithium, o-methylanilinide lithium, 2-methoxyanilinide lithium, 4-methoxy lithium anilinide, lithium 2,6,-dimethylanilinide, or lithium 2,6-diisopropylanilinide.

In the present invention, the lithium compound is a commercial lithium compound reagent.

In the above technical solution, wherein the borane is pinacolborane; and the carboxylic acid is acetic acid, caproic acid, valeric acid, heptanoic acid, trimethyl acetic acid, adipic acid, benzoic acid, 4-bromobenzoic acid, 4-fluorobenzoic acid, 1-naphthoic acid, 2-methoxybenzoic acid, o-carboxyphenylacetic acid, 3-indole acetic acid, 2-phenylbutyl Acid, 2-methyl-5-bromo-benzoic acid, 4-tert-butylbenzoic acid, 2-bromobenzoic acid, 4-iodobenzoic acid, 3-phenylpropionic acid or diphenylacetic acid.

In the above technical solution, the molar ratio of carboxylic acid to borane is 1:3 to 1:7.

In the above technical solution, the hydroboration is conducted at room temperature for 10 to 80 min. The amount of the lithium compound is 0.1% to 0.9% of molar amount of carboxylic acid.

Preferably, the lithium compound is n-butyllithium, the carboxylic acid is benzoic acid, 4-bromobenzoic acid, 4-fluorobenzoic acid, 1-naphthoic acid, 2-methoxybenzoic acid, o-carboxyphenylacetic acid, 3-indole acetic acid, 2-phenylbutyl Acid, 2-methyl-5-bromo-benzoic acid, 4-tert-butylbenzoic acid, 2-bromobenzoic acid, 4-iodobenzoic acid, 3-phenylpropionic acid or diphenylacetic acid. The hydroboration is conducted at room temperature for 40 to 50 min. The amount of the lithium compound is 0.4% to 0.6% of molar amount of carboxylic acid. Preferably, when the lithium compound is anilinolithium, p-toluidine lithium, o-methylanilinide lithium, 2-methoxyanilinide lithium, 4-methoxy lithium anilinide, lithium 2,6,-dimethylanilinide, or lithium 2,6-diisopropylanilinide, the carboxylic acid is benzoic acid, 4-bromobenzoic acid, 4-fluorobenzoic acid, 1-naphthoic acid, 2-methoxybenzoic acid, o-carboxyphenylacetic acid, 3-indole acetic acid, 2-phenylbutyl Acid, 2-methyl-5-bromo-benzoic acid, 4-tert-butylbenzoic acid, 2-bromobenzoic acid, 4-iodobenzoic acid, 3-phenylpropionic acid or diphenylacetic acid. The hydroboration is at room temperature for 70 to 80 min. The amount of the lithium compound is 0.7% to 0.9% of molar amount of carboxylic acid.

Preferably, when the lithium compound is n-butyllithium, the carboxylic acid is acetic acid, caproic acid, valeric acid, heptanoic acid, trimethyl acetic acid, adipic acid, the hydroboration is at room temperature for 10 to 20 min. The amount of the lithium compound is 0.1% to 0.3% of molar amount of carboxylic acid. Preferably, when the lithium compound is anilinolithium, p-toluidine lithium, o-methylanilinide lithium, 2-methoxyanilinide lithium, 4-methoxy lithium anilinide, lithium 2,6,-dimethylanilinide, or lithium 2,6-diisopropylanilinide, the carboxylic acid is acetic acid, caproic acid, valeric acid, heptanoic acid, trimethyl acetic acid, adipic acid, the hydroboration is at room temperature for 50 to 60 min. The amount of the lithium compound is 0.5% to 0.70% of molar amount of carboxylic acid.

In the above technical solution, after the hydroboration and is stopped by contacting air, the solvent is removed under reduced pressure, adding silica gel and methanol; after hydrolysis reaction, the solvent is removed under reduced pressure, then the alcohol compound is obtained by column chromatography.

In the above technical solution, the amount of carboxylic acid and silica gel, methanol is 1 mmol:2 to 2.2 g:6 mL; preferably, when the carboxylic acid is acetic acid, caproic acid, valeric acid, heptanoic acid, trimethylacetic acid, adipic acid, the amount of carboxylic acid and silica gel, methanol is 1 mmol:2.2 g:6 mL; when the carboxylic acid is benzoic acid, 4-bromobenzoic acid, 4-fluorobenzoic acid, 1-naphthoic acid, 2-methoxybenzoic acid, o-carboxyphenylacetic acid, 3-indole acetic acid, 2-phenylbutyl Acid, 2-methyl-5-bromo-benzoic acid, 4-tert-butylbenzoic acid, 2-bromobenzoic acid, 4-iodobenzoic acid, 3-phenylpropionic acid or diphenylacetic acid, the amount of carboxylic acid and silica gel, methanol is 1 mmol:2 g:6 mL.

In the above technical solution, hydrolysis reaction is at 50° C. for 115 to 120 min; preferably, when the carboxylic acid is acetic acid, caproic acid, valeric acid, heptanoic acid, trimethylacetic acid, adipic acid, the hydrolysis reaction is at 50° C. for 115 min; when the carboxylic acid is benzoic acid, 4-bromobenzoic acid, 4-fluorobenzoic acid, 1-naphthoic acid, 2-methoxybenzoic acid, o-carboxyphenylacetic acid, 3-indole acetic acid, 2-phenylbutyl Acid, 2-methyl-5-bromo-benzoic acid, 4-tert-butylbenzoic acid, 2-bromobenzoic acid, 4-iodobenzoic acid, 3-phenylpropionic acid or diphenylacetic acid, the hydrolysis reaction is at 50° C. for 120 min.

In the present invention, the specific steps of the method for preparing borate ester by hydroboration with carboxylic acid is: under the inert gas, stirring and mixing carboxylic acid and borane, and a catalyst lithium compound is added, then hydroboration; after the hydroboration and is stopped by contacting air, the solvent is removed under reduced pressure, to obtain the borate ester with different substitutents. The inert gas atmosphere can be realized in the glove box, which is a conventional technology.

At the room temperature, under the inert gas, stirring and mixing carboxylic acid and borane, and a catalyst lithium compound is added, then the borate ester is obtained with hydroboration; after hydroboration adding silica gel and methanol, the alcohol compound is obtained after hydrolysis reaction.

In the above technical solution, under the inert gas, stirring and mixing carboxylic acid and borane, and a catalyst lithium compound is added, then the borate ester is obtained with hydroboration; after hydroboration adding silica gel and methanol, the alcohol compound is obtained after hydrolysis reaction. The inert gas atmosphere can be realized in the glove box, which is a conventional technology.

In the present invention, all raw materials undergo anhydrous and oxygen-free treatment.

### Beneficial Effect

Due to the application of the above technical solutions, the present invention has the following advantages compared with the prior art:

1. The present invention finds for the first time that a commercial lithium compound can efficiently catalyze the borohydride reaction between carboxylic acid and borane, which is highly compatible with atom-economic synthesis.

2. The lithium compound disclosed in the present invention has high catalytic activity for catalyzing the borohydride reaction of carboxylic acid and borane (the amount of lithium compound is 0.1% to 0.9% of the molar amount of carboxylic acid), the reaction conditions are mild (room temperature), and the reaction time is short (10-50 minutes), and the reaction yield is high, the reaction is simple and controllable, and the post-treatment is simple.

3. The lithium compound-catalyzed borohydride of carboxylic acid disclosed in the present invention has a wide range of application to substrates, is suitable for carboxylic acids with different substituent positions and different electronic effects, and provides more options for the industrial synthesis of borate esters. The process is simple and controllable, the yield is high, the post-processing of the product is easy, and it is suitable for industrial production.

### DESCRIPTION OF THE DRAWINGS

FIG. 1 is the schematic diagram of the reaction in Example 1;

FIG. 2 is chemical structural formula of the 2,4-bis(2,6-diisopropylanilino)-2-pentenyllithium in comparative example.

### EMBODIMENTS OF THE INVENTION

The present invention will be further described in combination with the following embodiments:

### Example 1

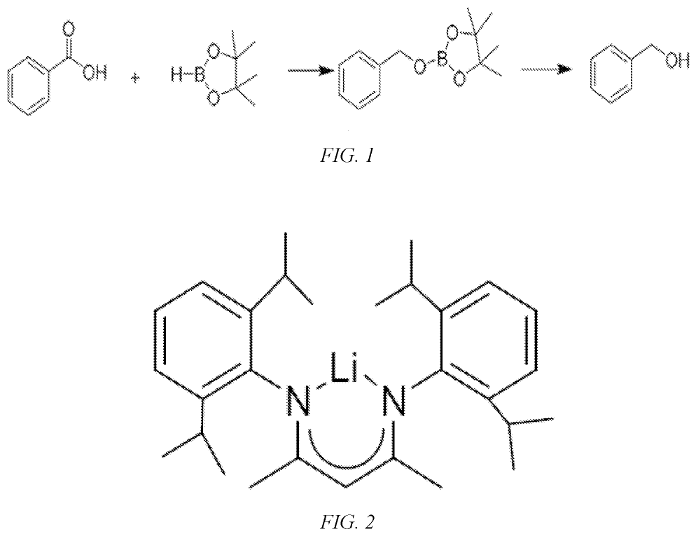

The schematic diagram of the reaction is shown in FIG. 1, and the reaction process in other embodiments is similar to this.

Under the protection of inert gas, loading the benzoic acid (61.1 mg, 0.5 mmol) to the reaction bottle, which is treated by dehydration and deoxidation, then adding a pinacolborane (218 μL, 1.5 mmol) with pipette gun, and finally adding 25 μL of tetrahydrofuran solution of n-butyl lithium (0.1M) (0.5 mol %, the same below), at room temperature for 45 min, then the hydroboration and is stopped by contacting air, the solvent is removed under reduced pressure, to obtain the borate ester. The mes-trimethoxybenzene (84.15 mg, 0.5 mmol) is used as the internal standard. The stock solution is dissolved in CDCl₃ stirring for 10 minutes, sampled, and equipped with nuclear magnetism. After calculation, conversion rate of ¹H is 97%, ¹H NMR analysis of the product borate ester is: ¹H NMR (400 MHz, CDCl₃): δ 7.22-7.32 (m, 5H, ArH), 4.92 (s, 2H, CH₂), 1.26 (s, 36H, CH₃); when the amount of the pinacolborane is 2 mmol, conversion rate of ¹H is 99%; when the amount of the pinacolborane is 361.2 μL, 2.5 mmol, conversion rate of ¹H is 99%. If with the triaryloxy rare earth catalyst Nd(OAr)₃(THF)₂, no product can be obtained instead.

Adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent is removed under reduced pressure, and purified by silica gel (100-200 mesh) column chromatography, washed with ethyl acetate/hexane (1:5 volume ratio) mixture, with a separation yield of 93%. ¹H NMR analysis of the product borate ester is: ¹H NMR (400 MHz, CDCl₃): δ 7.21-7.29 (m, 5H, ArH), 4.62 (s, 2H, CH₂), 1.87 (br s, 1H, OH).

### Comparative Example: The Hydroboration of Benzoic Acid and Pinacol Borane Catalyzed by 2,4-bis(2,6-diisopropylanilino)-2-pentenyllithium

Under the protection of inert gas, loading the benzoic acid (60.5 mg, 0.5 mmol) to the reaction bottle, which is treated by dehydration and deoxidation, then adding a pinacolborane (238 μL, 1.65 mmol) with pipette gun, and finally adding 25 μL of tetrahydrofuran solution of 2,4-bis(2,6-diisopropylanilino)-2-pentenyllithium (0.1M) (0.5 mol %), at room temperature for 45 min, then the hydroboration and is stopped by contacting air, the solvent is removed under reduced pressure, to obtain the borate ester. The mes-trimethoxybenzene (83.32 mg, 0.5 mmol) is used as the internal standard. The stock solution is dissolved in CDCl₃ stirring for 10 minutes, sampled, and equipped with nuclear magnetism. After calculation, conversion rate of ¹H is 12%, ¹H NMR analysis of the product borate ester is: ¹H NMR (400 MHz, CDCl₃): δ 7.22-7.32 (m, 5H, ArH), 4.92 (s, 2H, CH₂), 1.26 (s, 36H, CH₃). If the raw material is replaced with acetic acid (28.6 μL, 0.5 mmol) conversion rate of ¹H is 18%; ¹H NMR analysis of the product borate ester is: ¹H NMR (400 MHz, CDCl₃): δ 3.87 (q, 2H, CH₂), 1.24 (s, 36H, CH₃), 1.20 (br s, 3H, CH₃).

The chemical structural formula of the 2,4-bis(2,6-diisopropylanilino)-2-pentenyllithium in comparative example is shown in FIG. 2.

### Example 2

Under the protection of inert gas, loading the benzoic acid (61.1 mg, 0.5 mmol) to the reaction bottle, which is treated by dehydration and deoxidation, then adding a pinacolborane (218 μL, 1.5 mmol) with pipette gun, and finally adding 40 μL of tetrahydrofuran solution of aniline lithium (0.1M) (0.8 mol %, the same below), at room temperature for 75 min, then the hydroboration and is stopped by contacting air, the solvent is removed under reduced pressure, to obtain the borate ester. The mes-trimethoxybenzene (84.15 mg, 0.5 mmol) is used as the internal standard. The stock solution is dissolved in CDCl₃ stirring for 10 minutes, sampled, and equipped with nuclear magnetism. After calculation, conversion rate of ¹H is 99%. If with the triaryloxy rare earth catalyst Nd(OAr)₃(THF)₂, no product can be obtained instead. ¹H NMR analysis of the product borate ester is: ¹H NMR (400 MHz, CDCl₃): δ 7.20-7.30 (m, 5H, ArH), 4.91 (s, 2H, CH₂), 1.24 (s, 36H, CH₃); when the amount of the pinacolborane is 2 mmol, conversion rate of ¹H is 99%; when the amount of the pinacolborane is 361.2 μL, 2.5 mmol, conversion rate of ¹H is 99%.

The reaction is carried out at room temperature for 80 minutes, the yield is 99%.

### Example 3

Under the protection of inert gas, loading the 4-fluorobenzoic acid (70.8 mg, 0.5 mmol) to the reaction bottle, which is treated by dehydration and deoxidation, then adding a pinacolborane (290 μL, 2 mmol) with pipette gun, and finally adding tetrahydrofuran solution of aniline lithium (0.8 mol %), at room temperature for 75 min, then the hydroboration and is stopped by contacting air, the solvent is removed under reduced pressure, to obtain the borate ester. The mes-trimethoxybenzene (84.99 mg, 0.5 mmol) is used as the internal standard. The stock solution is dissolved in CDCl₃ stirring for 10 minutes, sampled, and equipped with nuclear magnetism. After calculation, conversion rate of ¹H is 91%. ¹H NMR analysis of the product borate ester is: ¹H NMR (400 MHz, CDCl₃): δ 7.20-7.30 (m, 5H, ArH), 4.91 (s, 2H, CH₂), 1.24 (s, 36H, CH₃).

### Example 4

Under the protection of inert gas, loading the 4-bromobenzoic acid (100 mg, 0.5 mmol) to the reaction bottle, which is treated by dehydration and deoxidation, then adding a pinacolborane (289 μL, 2 mmol) with pipette gun, and finally adding tetrahydrofuran solution of o-methylanilinide lithium (0.8 mol %), at room temperature for 75 min, then the hydroboration and is stopped by contacting air, the solvent is removed under reduced pressure, to obtain the borate ester. The mes-trimethoxybenzene (83.67 mg, 0.5 mmol) is used as the internal standard. The stock solution is dissolved in CDCl₃ stirring for 10 minutes, sampled, and equipped with nuclear magnetism. After calculation, conversion rate of ¹H is 93%. ¹H NMR analysis of the product borate ester is: ¹H NMR (400 MHz, CDCl₃): δ 7.39 (br s, 2H, ArCH), 7.16 (t, 2H, ArCH), 4.80 (s, 2H, OCH₂), 1.19 (s, 36H, CH₃).

### Example 5

Under the protection of inert gas, loading the 4-fluorobenzoic acid (70.8 mg, 0.5 mmol) to the reaction bottle, which is treated by dehydration and deoxidation, then adding a pinacolborane (290 μL, 2 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (84.99 mg, 0.5 mmol) is used as the internal standard. The stock solution is dissolved in CDCl₃ stirring for 10 minutes, sampled, and equipped with nuclear magnetism. After calculation, conversion rate of ¹H is 90%. ¹H NMR analysis of the product borate ester is: ¹H NMR (400 MHz, CDCl₃): δ 7.21 (br s, 2H, ArCH), 6.91 (t, 2H, ArCH), 4.75 (s, 2H, OCH₂), 1.15 (s, 36H, CH₃).

Adding 1 g of silica gel and 3 mL of methanol to the system where the solvent is removed after the hydroboration, and react at 50° C. for 2 h. After the reaction the product pure primary alcohol are extracted three times with ethyl acetate, and the organic layers were combined, dried with anhydrous sodium sulfate, the solvent is removed under reduced pressure, and purified by silica gel (100-200 mesh) column chromatography, washed with ethyl acetate/hexane (1:5 volume ratio) mixture, with a separation yield of 89%. ¹H NMR analysis of the product borate ester is: ¹H NMR (400 MHz, CDCl₃): δ 7.22 (br s, 2H, ArCH), 6.98 (t, 2H, ArCH), 4.56 (s, 2H, CH₂), 2.27 (br s, 1H, OH).

### Example 6

Under the protection of inert gas, loading the 4-bromobenzoic acid (100 mg, 0.5 mmol) to the reaction bottle, which is treated by dehydration and deoxidation, then adding a pinacolborane (289 μL, 2 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (83.67 mg, 0.5 mmol) is used as the internal standard. The stock solution is dissolved in CDCl₃ stirring for 10 minutes, sampled, and equipped with nuclear magnetism. After calculation, conversion rate of ¹H is 95%. ¹H NMR analysis of the product borate ester is: ¹H NMR (400 MHz, CDCl₃): δ 7.40 (br s, 2H, ArCH), 7.17 (t, 2H, ArCH), 4.81 (s, 2H, OCH₂), 1.20 (s, 36H, CH₃).

Adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent is removed under reduced pressure, and purified by silica gel (100-200 mesh) column chromatography, washed with ethyl acetate/hexane (1:5 volume ratio) mixture, with a separation yield of 91%. ¹H NMR analysis of the product borate ester is: ¹H NMR (400 MHz, CDCl₃): δ 7.42 (br s, 2H, ArCH), 7.19 (t, 2H, ArCH), 4.60 (s, 2H, CH₂), 2.26 (br s, 1H, OH).

### Example 7

Under the protection of inert gas, loading the 2-methoxybenzoic acid (76.2 mg, 0.5 mmol) to the reaction bottle, which is treated by dehydration and deoxidation, then adding a pinacolborane (290 μL, 2 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (84.23 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl₃): δ 7.41 (d, 1H, ArCH), 7.21 (t, 1H, ArCH), 6.95 (t, 1H, ArCH), 6.83 (d, 1H, ArCH), 4.97 (s, 2H, OCH₂), 1.26 (s, 36H, CH₃).

Adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent is removed under reduced pressure, and purified by silica gel (100-200 mesh) column chromatography, washed with ethyl acetate/hexane (1:5 volume ratio) mixture, with a separation yield of 91%. ¹H NMR analysis of the product borate ester is: ¹H NMR (400 MHz, CDCl₃): δ 7.42 (d, 1H, ArCH), 7.23 (t, 1H, ArCH), 6.96 (t, 1H, ArCH), 6.84 (d, 1H, ArCH), 4.67 (s, 2H, CH₂), 3.87 (br s, 1H, OH), 1.23 (s, 3H, CH₃).

### Example 8

Under the protection of inert gas, loading the 1-naphthoic acid (85.4 mg, 0.5 mmol) to the reaction bottle, which is treated by dehydration and deoxidation, then adding a pinacolborane (290 μL, 2 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (83.42 mg, 0.5 mmol) is used as the internal standard. The stock solution is dissolved in CDCl₃ stirring for 10 minutes, sampled, and equipped with nuclear magnetism. After calculation, conversion rate of ¹H is 91%. ¹H NMR analysis of the product borate ester is: ¹H NMR (400 MHz, CDCl₃): δ 8.01 (d, 1H, ArCH), 7.78-7.81 (m, 2H, ArCH), 7.74 (d, 1H, ArCH), 7.37-7.47 (m, 3H, ArCH), 5.36 (s, 2H, OCH₂), 1.22 (s, 36H, CH₃).

Adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 90%. ¹H NMR analysis of the product borate ester is: ¹H NMR (400 MHz, CDCl₃): δ 8.02 (d, 1H, ArCH), 7.80-7.82 (m, 2H, ArCH), 7.73 (d, 1H, ArCH), 7.38-7.48 (m, 3H, ArCH), 5.01 (s, 2H, CH₂), 2.31 (br s, 1H, OH).

### Example 9

Under the protection of inert gas, loading the 4-tert-butyl benzoic (88.9 mg, 0.5 mmol) to the reaction bottle, which is treated by dehydration and deoxidation, then adding a pinacolborane (290 μL, 2 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (83.89 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl₃): δ 7.27 (d, 2H, ArCH), 7.18 (d, 2H, ArCH), 4.81 (s, 2H, OCH₂), 1.22 (s, 9H, CH3, tBu), 1.17 (s, 36H, CH₃).

Adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 93%. ¹H NMR analysis of the product borate ester is: ¹H NMR (400 MHz, CDCl₃): δ 7.28 (d, 2H, ArCH), 7.16 (d, 2H, ArCH), 4.51 (s, 2H, CH₂), 2.12 (br s, 1H, OH), 1.23 (s, 9H, CH₃, tBu).

### Example 10

Under the protection of inert gas, loading the 2-bromobenzoic acid (100.6 mg, 0.5 mmol) to the reaction bottle, which is treated by dehydration and deoxidation, then adding a pinacolborane (290 μL, 2 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (84.17 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl₃): δ 7.40 (d, 2H, ArCH), 7.18-7.21 (m, 1H, ArCH), 7.02 (t, 1H, ArCH), 4.89 (s, 2H, OCH₂), 1.18 (s, 36H, CH₃).

Adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 91%. ¹H NMR analysis of the product borate ester is: ¹H NMR (400 MHz, CDCl₃): δ 7.41 (d, 2H, ArCH), 7.19-7.22 (m, 1H, ArCH), 7.03 (t, 1H, ArCH), 4.71 (s, 2H, CH₂), 2.41 (br s, 1H, OH).

### Example 11

Under the protection of inert gas, loading the 4-iodobenzoic acid (124.0 mg, 0.5 mmol) to the reaction bottle, which is treated by dehydration and deoxidation, then adding a pinacolborane (290 μL, 2 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (84.09 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl₃): δ 7.56 (d, 2H, ArCH), 7.01 (d, 2H, ArCH), 4.77 (s, 2H, OCH₂), 1.17 (s, 36H, CH₃).

Adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 91%. ¹H NMR analysis of the product borate ester is: ¹H NMR (400 MHz, CDCl₃): δ 7.57 (d, 2H, ArCH), 7.02 (d, 2H, ArCH), 4.65 (s, 2H, CH₂), 2.15 (br s, 1H, OH).

### Example 12

Under the protection of inert gas, loading the 3-phenylpropionic acid (74.9 mg, 0.5 mmol) to the reaction bottle, which is treated by dehydration and deoxidation, then adding a pinacolborane (289 μL, 2 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (83.89 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl₃): δ 7.17 (t, 2H, ArCH), 7.04-7.09 (m, 3H, ArCH), 3.79 (t, 2H, CH₂, OCH₂), 2.61 (t, 2H, CH₂), 1.75-1.82 (m, 2H, CH₂), 1.16 (s, 36H, CH₃).

Adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 95%. ¹H NMR analysis of the product borate ester is: ¹H NMR (400 MHz, CDCl3): δ 7.23 (t, 2H, ArCH), 7.11-7.13 (m, 3H, ArCH), 3.62 (t, 2H, CH₂, OCH₂), 2.65 (t, 2H, CH₂), 1.78-1.85 (m, 2H, CH₂), 1.61 (br s, 1H, OH).

### Example 13

Under the protection of inert gas, loading the diphenylacetic acid (105.8 mg, 0.5 mmol) to the reaction bottle, which is treated by dehydration and deoxidation, then adding a pinacolborane (289 μL, 2 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (83.84 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.13-7.32 (m, 10H, ArCH), 4.41 (d, 2H, CH₂, OCH₂), 4.24 (t, 1H, CH), 1.23 (s, 24H, CH₃, pinBOBpin), 1.12 (s, 12H, CH₃, OBpin).

Adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 91%. ¹H NMR analysis of the product borate ester is: ¹H NMR (400 MHz, CDCl3): δ 7.20-7.31 (m, 10H, ArCH), 4.19 (t, 1H, CH), 4.13 (d, 2H, CH₂), 1.64-1.70 (t, 1H, OH).

### Example 14

Under the protection of inert gas, loading the 2-methyl-5-bromobenzoic acid (107.1 mg, 0.5 mmol) to the reaction bottle, which is treated by dehydration and deoxidation, then adding a pinacolborane (289 μL, 2 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (83.77 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 6.90 (d, 1H, ArCH), 7.19 (d, 1H, ArCH), 7.47 (s, 1H, ArCH), 4.77 (s, 2H, OCH₂), 2.12 (s, 3H, CH₃), 1.17 (s, 36H, CH₃).

Adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 95%. ¹H NMR analysis of the product borate ester is: ¹H NMR (400 MHz, CDCl3): δ 6.91 (d, 1H, ArCH), 7.22 (d, 1H, ArCH), 7.57 (s, 1H, ArCH), 4.44 (s, 2H, OCH₂), 2.15 (s, 3H, CH₃), 2.25 (br s, 1H, OH).

### Example 15

Under the protection of inert gas, loading the 2-phenylbutyric acid (82.2 mg, 0.5 mmol) to the reaction bottle, which is treated by dehydration and deoxidation, then adding a pinacolborane (290 μL, 2 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (84.20 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.15-7.19 (m, 2H, ArCH), 7.08-7.10 (m, 3H, ArCH), 3.83-3.93 (m, 2H, CH₂, OCH₂), 2.57-2.66 (m, 1H, CH), 1.70-1.79 (m, 1H, CH₂), 1.46-1.55 (m, 1H, CH₂), 1.16 (s, 36H, CH₃, OBpin & pinBOBpin), 0.74 (t, 3H, CH₃).

Adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 93%. ¹H NMR analysis of the product borate ester is: ¹H NMR (400 MHz, CDCl3): δ 7.17-7.30 (m, 5H, ArCH), 3.66-3.68 (m, 2H, CH₂, OCH₂), 2.64 (m, 1H, CH), 1.54-1.73 (m, 1H, CH₂), 1.87 (s, 1H, OH), 0.82 (t, 3H, CH₃).

### Example 16

Under the protection of inert gas, loading the 3-indole acetic acid (88.0 mg, 0.5 mmol) to the reaction bottle, which is treated by dehydration and deoxidation, then adding a pinacolborane (363 μL, 2.5 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (84.49 mg, 0.5 mmol) is used as the internal standard. The stock solution is dissolved in CDCl₃ stirring for 10 minutes, sampled, and equipped with nuclear magnetism. After calculation, conversion rate of ¹H is 95%. ¹H NMR analysis of the product borate ester is: ¹H NMR (400 MHz, CDCl3): δ 7.82 (d, 1H, ArCH), 7.45 (d, 1H, ArCH), 7.02-7.15 (m, 3H, ArCH), 4.06 (t, 2H, OCH₂), 2.90 (t, 2H, CH₂), 1.29 (s, 12H, CH₃, N-Bpin) 1.14 (s, 24H, CH₃, pinBOBpin), 1.06 (s, 12H, CH₃, OBpin).

Adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 91%. ¹H NMR analysis of the product borate ester is: ¹H NMR (400 MHz, CDCl3): δ 8.10 (s, 1H, NH), 7.83 (d, 1H, ArCH), 7.45 (d, 1H, ArCH), 7.03-7.15 (m, 3H, ArCH), 4.67 (t, 2H, OCH₂), 3.28 (t, 2H, CH₂), 1.90 (br s, 1H, OH).

### Example 17

Under the protection of inert gas, loading the o-carboxyphenylacetic acid (90.0 mg, 0.5 mmol) to the reaction bottle, which is treated by dehydration and deoxidation, then adding a pinacolborane (508 μL, 3.5 mmol) with pipette gun, and finally adding tetrahydrofuran solution of n-butyl lithium (0.5 mol %), at room temperature for 45 min, then the hydroboration and is stopped by contacting air, the solvent is removed under reduced pressure, to obtain the borate ester. The mes-trimethoxybenzene (84.02 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.33 (br s, 1H, ArCH), 7.11 (br s, 3H, ArCH), 4.90 (s, 2H, CH₂). 3.96 (t, 2H, CH₂), 2.86 (t, 2H, CH₂), 1.17 (s, 72H, CH₃, OBpin & pinBOBpin). Continuing hydrolyzing to obtain pure primary alcohol, adding 1 g of silica gel and 3 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent was removed under reduced pressure, and purified by silica gel (100-200 mesh) column chromatography, washed with ethyl acetate/hexane (1:5 volume ratio) mixture, with a separation yield of 93%. ¹H NMR analysis of the product borate ester is: ¹H NMR (400 MHz, CDCl3): δ 7.32 (br s, 1H, ArCH), 7.12 (br s, 3H, ArCH), 4.53 (s, 2H, CH₂), 3.76 (t, 2H, CH₂OH), 3.7 (br, 1H, OH), 3.1 (br s, 1H, OH), 2.86 (t, 2H, CH₂).

### Example 18

Under the protection of inert gas, loading the 2-methoxybenzoic acid (76.2 mg, 0.5 mmol) to the reaction bottle, which is treated by dehydration and deoxidation, then adding a pinacolborane (290 μL, 2 mmol) with pipette gun, and finally adding tetrahydrofuran solution of aniline lithium (0.8 mol %), at room temperature for 75 min, then the hydroboration and is stopped by contacting air, the solvent is removed under reduced pressure, to obtain the borate ester. The mes-trimethoxybenzene (84.23 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.40 (d, 1H, ArCH), 7.21 (t, 1H, ArCH), 6.94 (t, 1H, ArCH), 6.82 (d, 1H, ArCH), 4.96 (s, 2H, OCH₂), 1.25 (s, 36H, CH₃).

### Example 19

Under the protection of inert gas, loading the 1-naphthoic acid (85.4 mg, 0.5 mmol) to the reaction bottle, which is treated by dehydration and deoxidation, then adding a pinacolborane (289 μL, 2 mmol) with pipette gun, and finally adding tetrahydrofuran solution of aniline lithium (0.8 mol %), at room temperature for 75 min, then the hydroboration and is stopped by contacting air, the solvent is removed under reduced pressure, to obtain the borate ester. The mes-trimethoxybenzene (83.42 mg, 0.5 mmol) is used as the internal standard. The stock solution is dissolved in CDCl₃ stirring for 10 minutes, sampled, and equipped with nuclear magnetism. After calculation, conversion rate of ¹H is 93%. ¹H NMR analysis of the product borate ester is: ¹H NMR (400 MHz, CDCl₃): δ 8.01 (d, 1H, ArCH), 7.78-7.80 (m, 2H, ArCH), 7.73 (d, 1H, ArCH), 7.36-7.46 (m, 3H, ArCH), 5.35 (s, 2H, OCH₂), 1.21 (s, 36H, CH₃).

### Example 20

Under the protection of inert gas, loading the 4-tert-butyl benzoic acid (88.9 mg, 0.5 mmol) to the reaction bottle, which is treated by dehydration and deoxidation, then adding a pinacolborane (290 μL, 2 mmol) with pipette gun, and finally adding tetrahydrofuran solution of 2-methoxyanilinide lithium (0.8 mol %), at room temperature for 80 min, then the hydroboration and is stopped by contacting air, the solvent is removed under reduced pressure, to obtain the borate ester. The mes-trimethoxybenzene (83.89 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.25 (d, 2H, ArCH), 7.16 (d, 2H, ArCH), 4.79 (s, 2H, OCH₂), 1.20 (s, 9H, CH₃, tBu), 1.15 (s, 36H, CH₃).

### Example 21

Under the protection of inert gas, loading the 2-bromobenzoic acid (100.6 mg, 0.5 mmol) to the reaction bottle, which is treated by dehydration and deoxidation, then adding a pinacolborane (290 μL, 2 mmol) with pipette gun, and finally adding tetrahydrofuran solution of aniline lithium (0.8 mol %), at room temperature for 75 min, then the hydroboration and is stopped by contacting air, the solvent is removed under reduced pressure, to obtain the borate ester. The mes-trimethoxybenzene (84.17 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.39 (d, 2H, ArCH), 7.17-7.20 (m, 1H, ArCH), 7.01 (t, 1H, ArCH), 4.88 (s, 2H, OCH₂), 1.17 (s, 36H, CH₃).

### Example 22

Under the protection of inert gas, loading the 4-iodobenzoic acid (124.0 mg, 0.5 mmol) to the reaction bottle, which is treated by dehydration and deoxidation, then adding a pinacolborane (290 μL, 2 mmol) with pipette gun, and finally adding tetrahydrofuran solution of 4-lithium methoxyanilide (0.8 mol %), at room temperature for 75 min, then the hydroboration and is stopped by contacting air, the solvent is removed under reduced pressure, to obtain the borate ester. The mes-trimethoxybenzene (84.09 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.55 (d, 2H, ArCH), 7.01 (d, 2H, ArCH), 4.76 (s, 2H, OCH₂), 1.16 (s, 36H, CH₃).

### Example 23

Under the protection of inert gas, loading the 3-phenylpropionic acid (74.9 mg, 0.5 mmol) to the reaction bottle, which is treated by dehydration and deoxidation, then adding a pinacolborane (289 μL, 2 mmol) with pipette gun, and finally adding tetrahydrofuran solution of aniline lithium (0.8 mol %), at room temperature for 75 min, then the hydroboration and is stopped by contacting air, the solvent is removed under reduced pressure, to obtain the borate ester. The mes-trimethoxybenzene (83.89 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.16 (t, 2H, ArCH), 7.03-7.08 (m, 3H, ArCH), 3.78 (t, 2H, CH₂, OCH₂), 2.60 (t, 2H, CH₂), 1.74-1.81 (m, 2H, CH₂), 1.15 (s, 36H, CH₃).

### Example 24

Under the protection of inert gas, loading the diphenylacetic acid (105.8 mg, 0.5 mmol) to the reaction bottle, which is treated by dehydration and deoxidation, then adding a pinacolborane (289 μL, 2 mmol) with pipette gun, and finally adding tetrahydrofuran solution of aniline lithium (0.8 mol %), at room temperature for 75 min, then the hydroboration and is stopped by contacting air, the solvent is removed under reduced pressure, to obtain the borate ester. The mes-trimethoxybenzene (83.84 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.12-7.31 (m, 10H, ArCH), 4.40 (d, 2H, CH₂, OCH₂), 4.23 (t, 1H, CH), 1.22 (s, 24H, CH₃, pinBOBpin), 1.11 (s, 12H, CH₃, OBpin).

### Example 25

Under the protection of inert gas, loading the 2-methyl-5-bromo-benzoic acid (107.1 mg, 0.5 mmol) to the reaction bottle, which is treated by dehydration and deoxidation, then adding a pinacolborane (289 μL, 2 mmol) with pipette gun, and finally adding tetrahydrofuran solution of aniline lithium (0.8 mol %), at room temperature for 75 min, then the hydroboration and is stopped by contacting air, the solvent is removed under reduced pressure, to obtain the borate ester. The mes-trimethoxybenzene (83.77 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 6.91 (d, 1H, ArCH), 7.20 (d, 1H, ArCH), 7.48 (s, 1H, ArCH), 4.78 (s, 2H, OCH2), 2.13 (s, 3H, CH3), 1.18 (s, 36H, CH3).

### Example 26

Under the protection of inert gas, loading the 2-phenylbutyric acid (82.2 mg, 0.5 mmol) to the reaction bottle, which is treated by dehydration and deoxidation, then adding a pinacolborane (290 μL, 2 mmol) with pipette gun, and finally adding tetrahydrofuran solution of aniline lithium (0.8 mol %), at room temperature for 75 min, then the hydroboration and is stopped by contacting air, the solvent is removed under reduced pressure, to obtain the borate ester. The mes-trimethoxybenzene (84.20 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl₃): δ 7.14-7.18 (m, 2H, ArCH), 7.07-7.10 (m, 3H, ArCH), 3.82-3.92 (m, 2H, CH2, OCH2), 2.56-2.65 (m, 1H, CH), 1.70-1.80 (m, 1H, CH2), 1.45-1.54 (m, 1H, CH2), 1.15 (s, 36H, CH3, OBpin & pinBOBpin), 0.73 (t, 3H, CH3).

### Example 27

Under the protection of inert gas, loading the 3-indole acetic acid (88.0 mg, 0.5 mmol) to the reaction bottle, which is treated by dehydration and deoxidation, then adding a pinacolborane (363 μL, 2.5 mmol) with pipette gun, and finally adding tetrahydrofuran solution of aniline lithium (0.8 mol %), at room temperature for 75 min, then the hydroboration and is stopped by contacting air, the solvent is removed under reduced pressure, to obtain the borate ester. The mes-trimethoxybenzene (84.49 mg, 0.5 mmol) is used as the internal standard. The stock solution is dissolved in CDCl₃ stirring for 10 minutes, sampled, and equipped with nuclear magnetism. After calculation, conversion rate of ¹H is 96%. ¹H NMR analysis of the product borate ester is: ¹H NMR (400 MHz, CDCl3): δ 7.81 (d, 1H, ArCH), 7.44 (d, 1H, ArCH), 7.01-7.13 (m, 3H, ArCH), 4.05 (t, 2H, OCH₂), 2.89 (t, 2H, CH₂), 1.28 (s, 12H, CH₃, N-Bpin) 1.13 (s, 24H, CH₃, pinBOBpin), 1.05 (s, 12H, CH₃, OBpin).

### Example 28

Under the protection of inert gas, loading the o-carboxyphenylacetic acid (90.0 mg, 0.5 mmol) to the reaction bottle, which is treated by dehydration and deoxidation, then adding a pinacolborane (508 μL, 3.5 mmol) with pipette gun, and finally adding tetrahydrofuran solution of aniline lithium (0.8 mol %), at room temperature for 75 min, then the hydroboration and is stopped by contacting air, the solvent is removed under reduced pressure, to obtain the borate ester. The mes-trimethoxybenzene (84.02 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.32 (br s, 1H, ArCH), 7.10 (br s, 3H, ArCH), 4.89 (s, 2H, CH₂), 3.95 (t, 2H, CH₂), 2.85 (t, 2H, CH₂), 1.16 (s, 72H, CH₃, OBpin & pinBOBpin).

### Example 29

Under the protection of inert gas, loading the benzoic acid (61.1 mg, 0.5 mmol) to the reaction bottle, which is treated by dehydration and deoxidation, then adding a pinacolborane (218 μL, 1.5 mmol) with pipette gun, and finally adding 40 μL of tetrahydrofuran solution of lithium p-tolylamine (0.1M) (0.8 mol %), at room temperature for 70 min, then the hydroboration and is stopped by contacting air, the solvent is removed under reduced pressure, to obtain the borate ester. The mes-trimethoxybenzene (84.15 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.23-7.33 (m, 5H, ArH), 4.93 (s, 2H, CH₂), 1.27 (s, 36H, CH₃).

### Example 30

Under the protection of inert gas, loading the 3-indole acetic acid (88.0 mg, 0.5 mmol) to the reaction bottle, which is treated by dehydration and deoxidation, then adding a pinacolborane (363 μL, 2.5 mmol) with pipette gun, and finally adding tetrahydrofuran solution of 2,6-dimethylanilinolithium (0.8 mol %), at room temperature for 75 min, then the hydroboration and is stopped by contacting air, the solvent is removed under reduced pressure, to obtain the borate ester. The mes-trimethoxybenzene (84.49 mg, 0.5 mmol) is used as the internal standard. The stock solution is dissolved in CDCl₃ stirring for 10 minutes, sampled, and equipped with nuclear magnetism. After calculation, conversion rate of ¹H is 96%. ¹H NMR analysis of the product borate ester is: ¹H NMR (400 MHz, CDCl3): δ 7.86 (d, 1H, ArCH), 7.49 (d, 1H, ArCH), 7.06-7.18 (m, 3H, ArCH), 4.10 (t, 2H, OCH₂), 2.94 (t, 2H, CH₂), 1.33 (s, 12H, CH₃, N-Bpin) 1.18 (s, 24H, CH₃, pinBOBpin), 1.10 (s, 12H, CH₃, OBpin).

### Example 31

Under the protection of inert gas, loading the 2-phenylbutyric acid (82.2 mg, 0.5 mmol) to the reaction bottle, which is treated by dehydration and deoxidation, then adding a pinacolborane (290 μL, 2 mmol) with pipette gun, and finally adding tetrahydrofuran solution of 2,6-diisopropylanilinolithium (0.1M) (0.8 mol %), at room temperature for 75 min, then the hydroboration and is stopped by contacting air, the solvent is removed under reduced pressure, to obtain the borate ester. The mes-trimethoxybenzene (84.20 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.20-7.24 (m, 2H, ArCH), 7.13-7.15 (m, 3H, ArCH), 3.88-3.98 (m, 2H, CH₂, OCH₂), 2.62-2.71 (m, 1H, CH), 1.75-1.84 (m, 1H, CH₂), 1.51-1.60 (m, 1H, CH₂), 1.21 (s, 36H, CH₃, OBpin & pinBOBpin), 0.79 (t, 3H, CH₃).

### Example 32

Under the protection of inert gas, loading the 2-methyl-5-bromobenzoic acid (107.1 mg, 0.5 mmol) to the reaction bottle, which is treated by dehydration and deoxidation, then adding a pinacolborane (289 μL, 2 mmol) with pipette gun, and finally adding tetrahydrofuran solution of p-toluidine lithium (0.8 mol %), at room temperature for 75 min, then the hydroboration and is stopped by contacting air, the solvent is removed under reduced pressure, to obtain the borate ester. The mes-trimethoxybenzene (83.77 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 6.92 (d, 1H, ArCH), 7.21 (d, 1H, ArCH), 7.49 (s, 1H, ArCH), 4.79 (s, 2H, OCH₂), 2.14 (s, 3H, CH₃), 1.19 (s, 36H, CH₃).

### Example 33

Under the protection of inert gas, loading the o-carboxyphenylacetic acid (90.0 mg, 0.5 mmol) to the reaction bottle, which is treated by dehydration and deoxidation, then adding a pinacolborane (508 μL, 3.5 mmol) with pipette gun, and finally adding tetrahydrofuran solution of lithium anilinolide (0.8 mol %), at room temperature for 80 min, then the hydroboration and is stopped by contacting air, the solvent is removed under reduced pressure, to obtain the borate ester. The mes-trimethoxybenzene (84.02 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 7.36 (br s, 1H, ArCH), 7.14 (br s, 3H, ArCH), 4.93 (s, 2H, CH₂), 3.99 (t, 2H, CH₂), 2.89 (t, 2H, CH₂), 1.20 (s, 72H, CH₃, OBpin & pinBOBpin).

### Example 34

Under the protection of inert gas, loading the 1-naphthoic acid (85.4 mg, 0.5 mmol) to the reaction bottle, which is treated by dehydration and deoxidation, then adding a pinacolborane (289 μL, 2 mmol) with pipette gun, and finally adding tetrahydrofuran solution of lithium 2,6,-dimethylanilinide (0.7 mol %), at room temperature for 75 min, then the hydroboration and is stopped by contacting air, the solvent is removed under reduced pressure, to obtain the borate ester. The mes-trimethoxybenzene (83.42 mg, 0.5 mmol) is used as the internal standard. The stock solution is dissolved in CDCl₃ stirring for 10 minutes, sampled, and equipped with nuclear magnetism. After calculation, conversion rate of ¹H is 93%. ¹H NMR analysis of the product borate ester is: ¹H NMR (400 MHz, CDCl3): δ 8.05 (d, 1H, ArCH), 7.83-7.85 (m, 2H, ArCH), 7.78 (d, 1H, ArCH), 7.38-7.48 (m, 3H, ArCH), 5.37 (s, 2H, OCH₂), 1.26 (s, 36H, CH₃).

### Example 35

Under the protection of inert gas, loading the acetic acid (28.6 μL, 0.5 mmol) to the reaction bottle, which is treated by dehydration and deoxidation, then adding a pinacolborane (290 μL, 2 mmol) with pipette gun, and finally adding 10 μL tetrahydrofuran solution of n-butyl lithium (0.1M) (0.2 mol %), at room temperature for 15 min, then the hydroboration and is stopped by contacting air, the solvent is removed under reduced pressure, to obtain the borate ester. The mes-trimethoxybenzene (84.08 mg, 0.5 mmol) is used as the internal standard. The stock solution is dissolved in CDCl₃ stirring for 10 minutes, sampled, and equipped with nuclear magnetism. After calculation, conversion rate of ¹H is 99%. If the n-butyllithium is replaced with 2,4-bis(2,6-diisopropylanilino)-2-pentenyllithium, conversion rate of ¹H is 15%. ¹H NMR analysis of the product borate ester is: ¹H NMR (400 MHz, CDCl₃): δ 3.88 (q, 2H, CH₂), 1.25 (s, 36H, CH₃), 1.21 (br s, 3H, CH₃).

Adding 1.1 g of silica gel and 3 mL of methanol to the system where the solvent is removed after the hydroboration, and react at 50° C. for 115 min. After the reaction the product aliphatic alcohol compounds are extracted three times with ethyl acetate, and the organic layers were combined, dried with anhydrous sodium sulfate, the solvent is removed under reduced pressure, and purified by silica gel (100-200 mesh) column chromatography, washed with ethyl acetate/hexane (1:5 volume ratio) mixture, with a separation yield of 95%. ¹H NMR analysis of the product borate ester is: ¹H NMR (400 MHz, CDCl3): δ 3.69 (q, 2H, CH₂), 2.92 (br s, 1H, OH), 1.22 (br s, 3H, CH₃).

When pinacol borane (218 μL, 1.5 mmol), the yield of borate is 95%; when pinacol borane (363 μL, 2.5 mmol), the yield of borate is 99%; reaction time is 20 min, The borate yield is 99%; if the n-butyl lithium is replaced with the triaryloxy rare earth catalyst, Nd(OAr)₃(THF)₂, the product borate cannot be obtained.

### Example 36

Under the protection of inert gas, loading the valeric acid (54.38 μL, 0.5 mmol) to the reaction bottle, which is treated by dehydration and deoxidation, then adding a pinacolborane (290 μL, 2 mmol) with pipette gun, and finally adding tetrahydrofuran solution of n-butyl lithium (0.2 mol %), at room temperature for 15 min, then the hydroboration and is stopped by contacting air, the solvent is removed under reduced pressure, to obtain the borate ester. The mes-trimethoxybenzene (84.12 mg, 0.5 mmol) is used as the internal standard. The stock solution is dissolved in CDCl₃ stirring for 10 minutes, sampled, and equipped with nuclear magnetism. After calculation, conversion rate of ¹H is 92%. ¹H NMR analysis of the product borate ester is: ¹H NMR (400 MHz, CDCl₃): δ 3.81 (t, 2H, OCH₂), 1.52-1.56 (m, 2H, CH₂), 1.30-1.52 (m, 4H, CH₂), 1.28 (s, 36H, CH), 0.86 (t, 3H, CH₃).

Adding 1.1 g of silica gel and 3 mL of methanol to the system where the solvent is removed after the hydroboration, and react at 50° C. for 115 min. After the reaction the product aliphatic alcohol compounds are extracted three times with ethyl acetate, and the organic layers were combined, dried with anhydrous sodium sulfate, the solvent is removed under reduced pressure, and purified by silica gel (100-200 mesh) column chromatography, washed with ethyl acetate/hexane (1:5 volume ratio) mixture, with a separation yield of 92%. ¹H NMR analysis of the product borate ester is: ¹H NMR (400 MHz, CDCl3): δ 3.63 (t, 2H, OCH₂), 1.59 (m, 2H, CH₂), 1.35 (m, 2H, CH₂), 2.35 (br s, 1H, OH), 0.90 (t, 3H, CH₃).

### Example 37

Under the protection of inert gas, loading the caproic acid (62.52 μL, 0.5 mmol) to the reaction bottle, which is treated by dehydration and deoxidation, then adding a pinacolborane (290 μL, 2 mmol) with pipette gun, and finally adding tetrahydrofuran solution of n-butyl lithium (0.2 mol %), at room temperature for 15 min, then the hydroboration and is stopped by contacting air, the solvent is removed under reduced pressure, to obtain the borate ester. The mes-trimethoxybenzene (84.01 mg, 0.5 mmol) is used as the internal standard. The stock solution is dissolved in CDCl₃ stirring for 10 minutes, sampled, and equipped with nuclear magnetism. After calculation, conversion rate of ¹H is 90%. ¹H NMR analysis of the product borate ester is: ¹H NMR (400 MHz, CDCl3): δ 3.76 (t, 2H, OCH₂), 1.46-1.52 (m, 2H, CH₂), 1.24-1.35 (m, 6H, CH₂), 1.19 (s, 48H, CH₃), 0.82 (t, 3H, CH₃).

Adding 1.1 g of silica gel and 3 mL of methanol to the system where the solvent is removed after the hydroboration, and react at 50° C. for 115 min. After the reaction the product aliphatic alcohol compounds are extracted three times with ethyl acetate, and the organic layers were combined, dried with anhydrous sodium sulfate, the solvent is removed under reduced pressure, and purified by silica gel (100-200 mesh) column chromatography, washed with ethyl acetate/hexane (1:5 volume ratio) mixture, with a separation yield of 89%. ¹H NMR analysis of the product borate ester is: ¹H NMR (400 MHz, CDCl3): δ 3.75 (t, 2H, OCH₂), 1.47-1.52 (m, 2H, CH₂), 1.26-1.36 (m, 6H, CH₂), 1.71 (br s, 1H, OH₃), 0.82 (t, 3H, CH₃).

### Example 38

Under the protection of inert gas, loading the heptanoic acid (70.90 μL, 0.5 mmol) to the reaction bottle, which is treated by dehydration and deoxidation, then adding a pinacolborane (290 μL, 2 mmol) with pipette gun, and finally adding tetrahydrofuran solution of n-butyl lithium (0.2 mol %), at room temperature for 15 min, then the hydroboration and is stopped by contacting air, the solvent is removed under reduced pressure, to obtain the borate ester. The mes-trimethoxybenzene (84.05 mg, 0.5 mmol) is used as the internal standard. The stock solution is dissolved in CDCl₃ stirring for 10 minutes, sampled, and equipped with nuclear magnetism. After calculation, conversion rate of ¹H is 90%. ¹H NMR analysis of the product borate ester is: ¹H NMR (400 MHz, CDCl3): δ 3.71 (t, 2H, OCH₂), 1.41-1.47 (m, 2H, CH₂), 1.19-1.30 (m, 8H, CH₂), 1.24 (s, 48H, CH₃), 0.77 (t, 3H, CH₃).

Adding 1.1 g of silica gel and 3 mL of methanol to the system where the solvent is removed after the hydroboration, and react at 50° C. for 115 min. After the reaction the product aliphatic alcohol compounds are extracted three times with ethyl acetate, and the organic layers were combined, dried with anhydrous sodium sulfate, the solvent is removed under reduced pressure, and purified by silica gel (100-200 mesh) column chromatography, washed with ethyl acetate/hexane (1:5 volume ratio) mixture, with a separation yield of 88%. ¹H NMR analysis of the product borate ester is: ¹H NMR (400 MHz, CDCl3): δ 3.72 (t, 2H, OCH₂), 1.42-1.47 (m, 2H, CH₂), 1.20-1.30 (m, 8H, CH₂), 1.76 (br s, 1H, OH₃), 0.77 (t, 3H, CH₃).

### Example 39

Under the protection of inert gas, loading the trimethylacetic acid (50.7 mg, 0.5 mmol) to the reaction bottle, which is treated by dehydration and deoxidation, then adding a pinacolborane (288 μL, 2 mmol) with pipette gun, and finally adding tetrahydrofuran solution of n-butyl lithium (0.2 mol %), at room temperature for 15 min, then the hydroboration and is stopped by contacting air, the solvent is removed under reduced pressure, to obtain the borate ester. The mes-trimethoxybenzene (84.08 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 3.43 (s, 2H, OCH₂), 1.17 (s, 36H, CH₃, OBpin & pinBOBpin), 0.82 (s, 9H, CH₃).

Adding 1.1 g of silica gel and 3 mL of methanol to the system where the solvent is removed after the hydroboration, and react at 50° C. for 115 min. After the reaction the product aliphatic alcohol compounds are extracted three times with ethyl acetate, and the organic layers were combined, dried with anhydrous sodium sulfate, the solvent is removed under reduced pressure, and purified by silica gel (100-200 mesh) column chromatography, washed with ethyl acetate/hexane (1:5 volume ratio) mixture, with a separation yield of 92%. ¹H NMR analysis of the product borate ester is: ¹H NMR (400 MHz, CDCl3): δ 3.20 (s, 2H, OCH₂), 1.97 (br s, 1H, OH), 0.81 (s, 9H, CH₃).

### Example 40

Under the protection of inert gas, loading the adipic acid (72.9 mg, 0.5 mmol) to the reaction bottle, which is treated by dehydration and deoxidation, then adding a pinacolborane (508 μL, 3.5 mmol) with pipette gun, and finally adding tetrahydrofuran solution of n-butyl lithium (0.2 mol %), at room temperature for 15 min, then the hydroboration and is stopped by contacting air, the solvent is removed under reduced pressure, to obtain the borate ester. The mes-trimethoxybenzene (83.90 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl3): δ 3.77 (t, 4H, OCH₂), 1.48-1.50 (m, 4H, CH₂), 1.28-1.30 (m, 4H, CH₂), 1.17 (s, 72H, CH₃, OBpin & pinBOBpin).

Adding 1.1 g of silica gel and 3 mL of methanol to the system where the solvent is removed after the hydroboration, and react at 50° C. for 115 min. After the reaction the product aliphatic alcohol compounds are extracted three times with ethyl acetate, and the organic layers were combined, dried with anhydrous sodium sulfate, the solvent is removed under reduced pressure, and purified by silica gel (100-200 mesh) column chromatography, washed with ethyl acetate/hexane (1:5 volume ratio) mixture, with a separation yield of 92%. ¹H NMR analysis of the product borate ester is: ¹H NMR (400 MHz, CDCl₃): δ 3.76 (t, 4H, OCH₂), 1.48-1.50 (m, 4H, CH₂), 1.29-1.31 (m, 4H, CH₂), 2.25 (br s, 2H, OH).

### Example 41

Under the protection of inert gas, loading the acetic acid (28.6 μL, 0.5 mmol) to the reaction bottle, which is treated by dehydration and deoxidation, then adding a pinacolborane (290 μL, 2 mmol) with pipette gun, and finally adding 30 μL tetrahydrofuran solution of aniline lithium (0.1M) (0.6 mol %, the same below), at room temperature for 55 min, then the hydroboration and is stopped by contacting air, the solvent is removed under reduced pressure, to obtain the borate ester. The mes-trimethoxybenzene (84.08 mg, 0.5 mmol) is used as the internal standard. The stock solution is dissolved in CDCl₃ stirring for 10 minutes, sampled, and equipped with nuclear magnetism. After calculation, conversion rate of ¹H is 99%. ¹H NMR analysis of the product borate ester is: ¹H NMR (400 MHz, CDCl₃): δ 3.86 (q, 2H, CH₂), 1.24 (s, 36H, CH₃), 1.20 (br s, 3H, CH₃).

When pinacol borane (218 μL, 1.5 mmol), the yield of borate is 96%; when pinacol borane (363 μL, 2.5 mmol), the yield of borate is 99%; reaction time is 30 min, The borate yield is 99%; if the n-butyl lithium is replaced with the triaryloxy rare earth catalyst, Nd(OAr)₃(THF)₂, the product borate cannot be obtained. If anilinolithium is replaced with p-toluidine lithium, o-toluidine lithium, 2-methoxy lithium anilide, lithium 4-methoxyanilide, lithium 2,6,-dimethylanilide or lithium 2,6-diisopropylanilide, the conversion rates of ¹H are all 99%.

### Example 42

Under the protection of inert gas, loading the valeric acid (54.38 μL, 0.5 mmol) to the reaction bottle, which is treated by dehydration and deoxidation, then adding a pinacolborane (290 μL, 2 mmol) with pipette gun, and finally adding tetrahydrofuran solution of aniline lithium (0.6 mol %), at room temperature for 55 min, then the hydroboration and is stopped by contacting air, the solvent is removed under reduced pressure, to obtain the borate ester. The mes-trimethoxybenzene (84.12 mg, 0.5 mmol) is used as the internal standard. The stock solution is dissolved in CDCl₃ stirring for 10 minutes, sampled, and equipped with nuclear magnetism. After calculation, conversion rate of ¹H is 92%. ¹H NMR analysis of the product borate ester is: ¹H NMR (400 MHz, CDCl₃): δ 3.80 (t, 2H, OCH₂), 1.51-1.55 (m, 2H, CH₂), 1.29-1.51 (m, 4H, CH₂), 1.27 (s, 36H, CH), 0.85 (t, 3H, CH₃).

### Example 43

Under the protection of inert gas, loading the hexanoic acid (62.52 μL, 0.5 mmol) to the reaction bottle, which is treated by dehydration and deoxidation, then adding a pinacolborane (290 μL, 2 mmol) with pipette gun, and finally adding 30 μL tetrahydrofuran solution of aniline lithium (0.6 mol %), at room temperature for 55 min, then the hydroboration and is stopped by contacting air, the solvent is removed under reduced pressure, to obtain the borate ester. The mes-trimethoxybenzene (84.01 mg, 0.5 mmol) is used as the internal standard. The stock solution is dissolved in CDCl₃ stirring for 10 minutes, sampled, and equipped with nuclear magnetism. After calculation, conversion rate of ¹H is 90%.

If anilinolithium is replaced with p-toluidine lithium, o-toluidine lithium, 2-methoxy Lithium anilide, lithium 4-methoxyanilide, lithium 2,6,-dimethylanilide or lithium 2,6-diisopropylanilide, the conversion rates of ¹H is 91%, 90%, 93%, 90%, 91%, 92%.

¹H NMR analysis of the product borate ester is: ¹H NMR (400 MHz, CDCl3): δ 3.75 (t, 2H, OCH₂), 1.45-1.51 (m, 2H, CH₂), 1.23-1.34 (m, 6H, CH₂), 1.18 (s, 48H, CH₃), 0.81 (t, 3H, CH₃).

### Example 44

Under the protection of inert gas, loading the heptanoic acid (70.90 μL, 0.5 mmol) to the reaction bottle, which is treated by dehydration and deoxidation, then adding a pinacolborane (290 μL, 2 mmol) with pipette gun, and finally adding tetrahydrofuran solution of aniline lithium (0.6 mol %), at room temperature for 55 min, then the hydroboration and is stopped by contacting air, the solvent is removed under reduced pressure, to obtain the borate ester. The mes-trimethoxybenzene (84.05 mg, 0.5 mmol) is used as the internal standard. The stock solution is dissolved in CDCl₃ stirring for 10 minutes, sampled, and equipped with nuclear magnetism. After calculation, conversion rate of ¹H is 90%. ¹H NMR analysis of the product borate ester is: ¹H NMR (400 MHz, CDCl3): δ 3.70 (t, 2H, OCH₂), 1.40-1.46 (m, 2H, CH₂), 1.18-1.29 (m, 8H, CH₂), 1.23 (s, 48H, CH₃), 0.76 (t, 3H, CH₃).

### Example 45

Under the protection of inert gas, loading the trimethylacetic acid (50.7 mg, 0.5 mmol) to the reaction bottle, which is treated by dehydration and deoxidation, then adding a pinacolborane (289 μL, 2 mmol) with pipette gun, and finally adding tetrahydrofuran solution of aniline lithium (0.6 mol %), at room temperature for 55 min, then the hydroboration and is stopped by contacting air, the solvent is removed under reduced pressure, to obtain the borate ester. The mes-trimethoxybenzene (84.08 mg, 0.5 mmol) is used as the internal standard. The stock solution is dissolved in CDCl₃ stirring for 10 minutes, sampled, and equipped with nuclear magnetism. After calculation, conversion rate of ¹H is 99%. If anilinolithium is replaced with p-toluidine lithium, o-toluidine lithium, 2-methoxy lithium anilide, lithium 4-methoxyanilide, lithium 2,6,-dimethylanilide or lithium 2,6-diisopropylanilide, the conversion rates of ¹H is 96%, 99%, 98%, 99%, 99%, 97%.

¹H NMR analysis of the product borate ester is: ¹H NMR (400 MHz, CDCl3): δ 3.42 (s, 2H, OCH₂), 1.16 (s, 36H, CH₃, OBpin & pinBOBpin), 0.81 (s, 9H, CH₃).

### Example 46

Under the protection of inert gas, loading the adipic acid (72.9 mg, 0.5 mmol) to the reaction bottle, which is treated by dehydration and deoxidation, then adding a pinacolborane (508 μL, 3.5 mmol) with pipette gun, and finally adding tetrahydrofuran solution of aniline lithium (0.6 mol %), at room temperature for 55 min, then the hydroboration and is stopped by contacting air, the solvent is removed under reduced pressure, to obtain the borate ester. The mes-trimethoxybenzene (83.90 mg, 0.5 mmol) is used as the internal standard. The stock solution is dissolved in CDCl₃ stirring for 10 minutes, sampled, and equipped with nuclear magnetism. After calculation, conversion rate of ¹H is 99%. If anilinolithium is replaced with p-toluidine lithium, o-toluidine lithium, 2-methoxy lithium anilide, lithium 4-methoxyanilide, lithium 2,6,-dimethylanilide or lithium 2,6-diisopropylanilide, the conversion rates of ¹H is 99%, 99%, 98%, 99%, 97%, 96%.

¹H NMR analysis of the product borate ester is: ¹H NMR (400 MHz, CDCl3): δ 3.76 (t, 4H, OCH₂), 1.47-1.49 (m, 4H, CH₂), 1.27-1.29 (m, 4H, CH₂), 1.16 (s, 72H, CH₃, OBpin & pinBOBpin).

### Example 47

Under the protection of inert gas, loading the benzoic acid (50 mmol) to the reaction bottle, which is treated by dehydration and deoxidation, then adding a pinacolborane (200 mmol) with pipette gun, and finally adding tetrahydrofuran solution of n-butyl lithium (0.1M) (0.5 mol %), at room temperature for 45 min, then the hydroboration and is stopped by contacting air, the solvent is removed under reduced pressure, to obtain the borate ester. The mes-trimethoxybenzene (84.15 mg, 0.5 mmol) is used as the internal standard. The stock solution is dissolved in CDCl₃ stirring for 10 minutes, sampled, and equipped with nuclear magnetism. After calculation, conversion rate of ¹H is 99%.

Adding 100 g of silica gel and 300 mL of methanol to the system where the solvent is removed after the hydroboration, and react at 50° C. for 2 h. After the reaction the product pure primary alcohol is extracted three times with ethyl acetate, and the organic layers were combined, dried with anhydrous sodium sulfate, the solvent is removed under reduced pressure, and purified by silica gel (100-200 mesh) column chromatography, washed with ethyl acetate/hexane (1:5 volume ratio) mixture, with a separation yield of 94%.

### Example 48

Under the protection of inert gas, loading the acetic acid (50 mmol) to the reaction bottle, which is treated by dehydration and deoxidation, then adding a pinacolborane (200 mmol) with pipette gun, and finally adding tetrahydrofuran solution of n-butyl lithium (0.1M) (0.2 mol %), at room temperature for 15 min, then the hydroboration and is stopped by contacting air, the solvent is removed under reduced pressure, to obtain the borate ester. The mes-trimethoxybenzene (84.08 mg, 0.5 mmol) is used as the internal standard. The stock solution is dissolved in CDCl₃ stirring for 10 minutes, sampled, and equipped with nuclear magnetism. After calculation, conversion rate of ¹H is 99%.

Adding 110 g of silica gel and 300 mL of methanol to the system where the solvent is removed after the hydroboration, and react at 50° C. for 115 min. After the reaction the product pure primary alcohol is extracted three times with ethyl acetate, and the organic layers were combined, dried with anhydrous sodium sulfate, the solvent is removed under reduced pressure, and purified by silica gel (100-200 mesh) column chromatography, washed with ethyl acetate/hexane (1:5 volume ratio) mixture, with a separation yield of 95%.

1. A method for preparing a borate ester based using a lithium compound comprising the following steps: under inert gas, stirring and mixing a carboxylic acid and a borane, adding the lithium compound as a catalyst is added, and conducting a hydroboration to obtain the borate ester; wherein the lithium compound is n-butyllithium, anilinolithium, p-toluidine lithium, o-methylanilinide lithium, 2-methoxyanilinide lithium, 4-methoxy lithium anilinide, lithium 2,6,-dimethylanilinide, or lithium 2,6-diisopropylanilinide.

2. The method according to claim 1, wherein the borane is pinacolborane; and the carboxylic acid is acetic acid, caproic acid, valeric acid, heptanoic acid, trimethyl acetic acid, adipic acid, benzoic acid, 4-bromobenzoic acid, 4-fluorobenzoic acid, 1-naphthoic acid, 2-methoxybenzoic acid, o-carboxyphenylacetic acid, 3-indole acetic acid, 2-phenylbutyl Acid, 2-methyl-5-bromo-benzoic acid, 4-tert-butylbenzoic acid, 2-bromobenzoic acid, 4-iodobenzoic acid, 3-phenylpropionic acid, or diphenylacetic acid.

3. The method according to claim 1, wherein a molar ratio of the carboxylic acid to the borane is 1:3 to 1:7; and an amount of the lithium compound is 0.1% to 0.9% of a molar amount of the carboxylic acid.

4. The method according to claim 1, wherein the hydroboration is conducted at room temperature for 10 to 80 min.

5. The method according to claim 1, wherein the hydroboration is stopped by contacting air, a solvent is removed under reduced pressure to obtain the borate ester.

6. A method for preparing an alcohol compound using on lithium compound, comprising the following steps: under inert gas, stirring and mixing a carboxylic acid and a borane, adding the lithium compound as a catalyst, and conducting a hydroboration to obtain the borate ester; after the hydroboration, adding silica gel and methanol, conducting a hydrolysis reaction to obtain the alcohol compound, wherein the lithium compound is n-butyllithium, aniline lithium, p-toluidine lithium, o-methylanilinide lithium, 2-methoxyanilinide lithium, 4-methoxy lithium anilinide, lithium 2,6,-dimethylanilinide, or lithium 2,6-diisopropylanilinide.

7. The method according to claim 6, wherein the hydroboration is stopped by contacting air, a solvent is removed under reduced pressure; silica gel and methanol are added; after the hydrolysis reaction, a solvent is removed under reduced pressure, and the alcohol compound is obtained by column chromatography.

8. The method according to claim 6, wherein a ratio of carboxylic acid and silica gel, and methanol is 1 mmol:2 to 2.2 g:6 mL; a molar ratio of the carboxylic acid to borane is 1:3 to 1:7; and an amount of the lithium compound is 0.1% to 0.9% of a molar amount of the carboxylic acid.

9. The method according to claim 6, wherein hydrolysis reaction is conducted at 50° C. for 115 to 120 min; the hydroboration is conducted at room temperature for 10 to 80 min.

10. The method according to claim 6, wherein the borane is pinacolborane; and the carboxylic acid is acetic acid, caproic acid, valeric acid, heptanoic acid, trimethyl acetic acid, adipic acid, benzoic acid, 4-bromobenzoic acid, 4-fluorobenzoic acid, 1-naphthoic acid, 2-methoxybenzoic acid, o-carboxyphenylacetic acid, 3-indole acetic acid, 2-phenylbutyl acid, 4-iodobenzoic acid, 3-phenylpropionic acid, diphenylacetic acid, 2-phenylbutyric acid, indole-3-acetic acid, o-carboxyphenylacetic acid, or 2-methyl-5-bromobenzoic acid.
